(19) United States
(12) Plant Patent Application Publication
(10) Pub. No.: US 2014/0373231 P1
(43) Pub. Date: Dec. 18, 2014

(54) Clematis Plant Named 'Evipo046'

(50) ;

(71) Applicants: Mogens Nyegaard Olesen, Fredensborg (DK); Raymond Evison, St. Sampsons (GB)

(72) Inventors: Mogens Nyegaard Olesen, Fredensborg (DK); Raymond Evison, St. Sampsons (GB)

(21) Appl. No.: 13/986,923

(22) Filed: Jun. 14, 2013

(51) Int. Cl. A01H 5/00

(52) U.S. Cl. PLT/228

(57) ABSTRACT

A new Clematis plant with a compact growth habit, profuse, violet flowers, and continuous summer flowering. The variety successfully propagates from softwood cuttings and is suitable for cultivation commercial nursery culture. This new and distinct variety has shown to be uniform and stable in the resulting generations from asexual propagation from vegetative cuttings.

### BOTANICAL CLASSIFICATION

Genus: Clematis

Species: viticella

### VARIETY DENOMINATION

‘Evipo046’

### SUMMARY OF THE CLAIMED PLANT

The present invention constitutes a new and distinct variety of Clematis plant which originated from a controlled crossing between the female seed parent, an un-named seedling, and the male pollen parent, an un-named seedling

The two parents were crossed during the summer of 1996 and the resulting seeds were planted the following winter in a controlled environment in Guernsey, Channel Islands. The new variety named ‘Evipo046’ originated as a single seedling from the stated cross.

The new clematis plant may be distinguished from its female seed parent and male pollen parent primarily by flower color and growth habit.

The objective of the hybridization of this clematis plant was to create a new and distinct variety for nursery culture with unique qualities such as:
1. Uniform and abundant violet flowers;
2. Vigorous and compact growth, making the variety suitable for container culture
3. Improved disease resistance; and
4. Deep purple center.

This combination of qualities was lacking in clematis plants that were in commercial cultivation and the qualities have been substantially achieved in the new variety.

‘Evipo046’ was selected by Mogens N. Olesen and Raymond J. Evison in their clematis development program in the Channel Islands, United Kingdom in 1997. Asexual reproduction of ‘Evipo046’ by means of vegetative cuttings and traditional layering was first performed by Mogens N. Olesen and Raymond J. Evison in the nursery during the summer of 1997. This initial and subsequent asexual propagations have demonstrated that the characteristics of ‘Evipo046’ are true to type and are transmitted from one generation to the next.

### BRIEF DESCRIPTION OF THE DRAWING

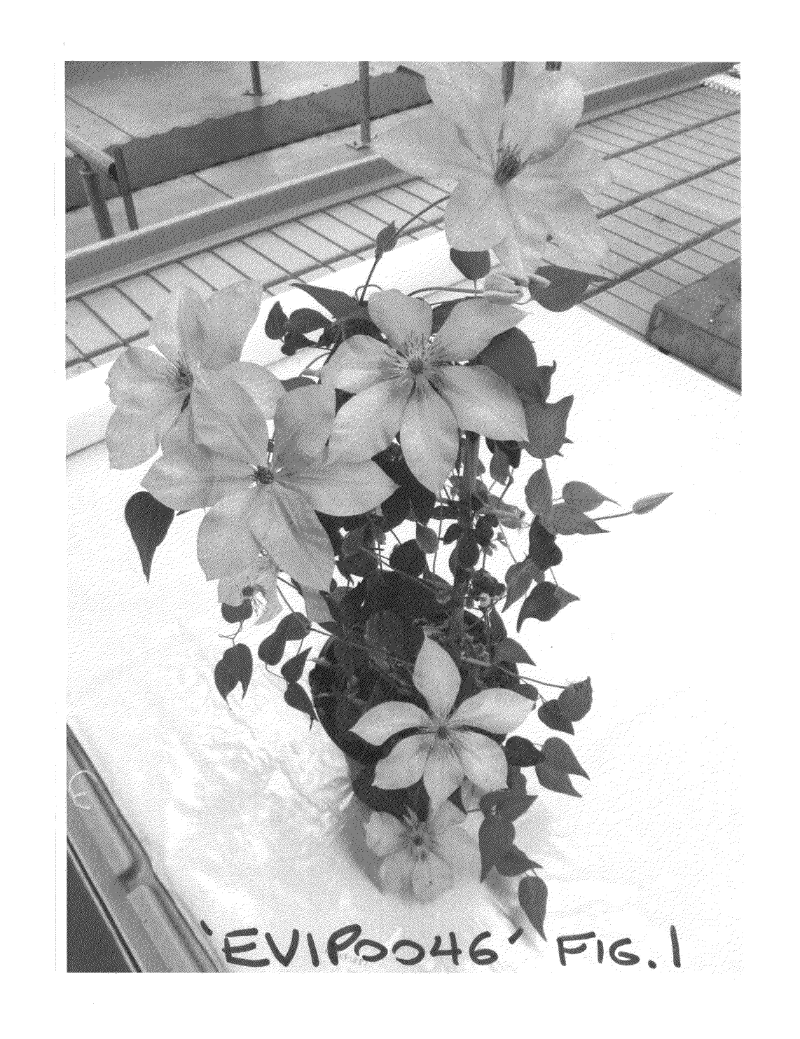

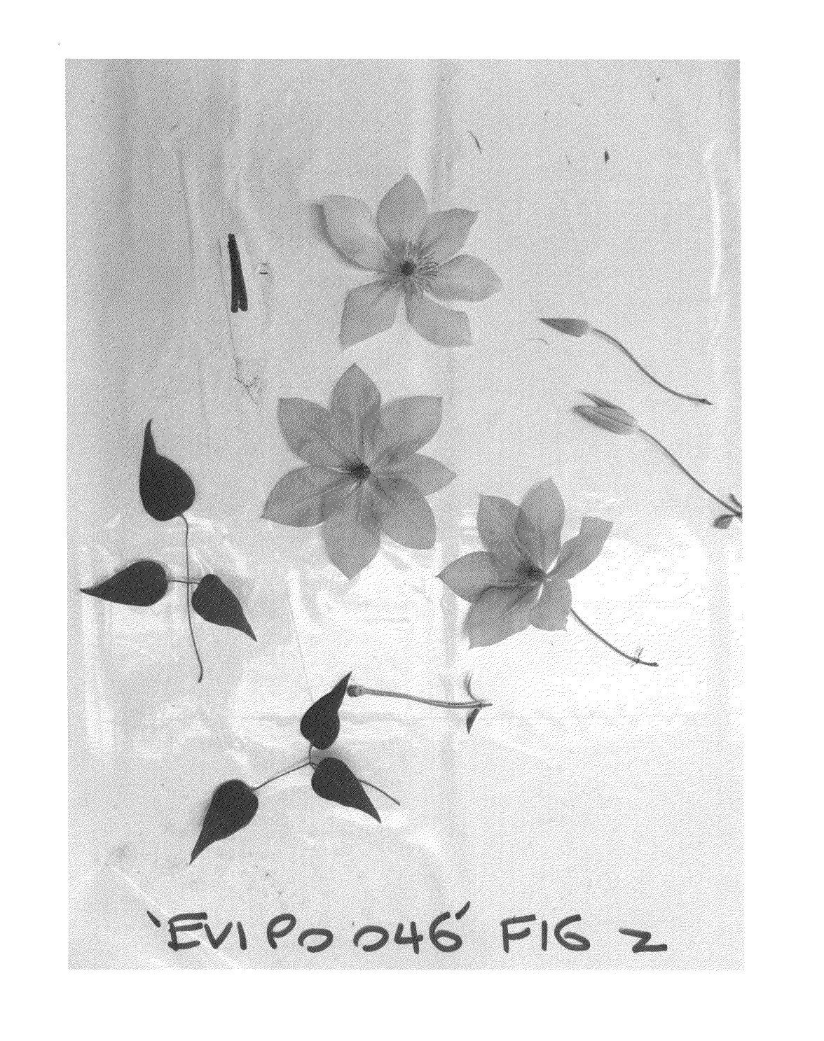

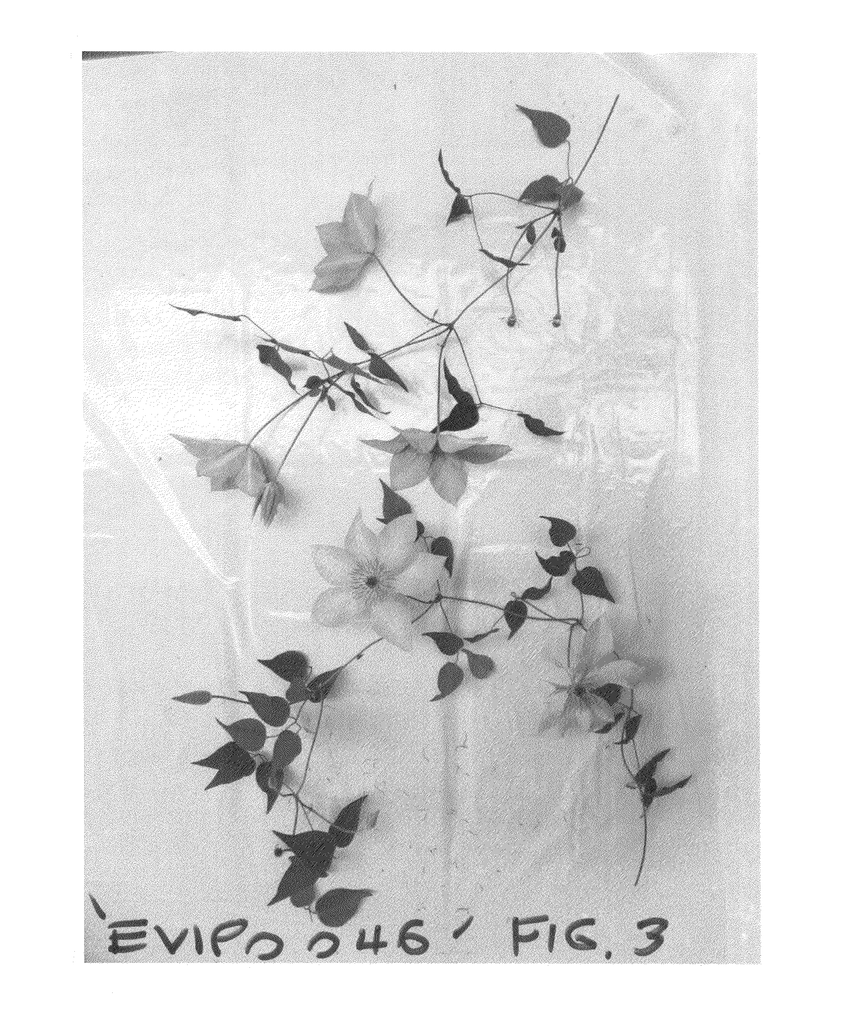

The accompanying color illustrations show as true as is reasonably possible to obtain in color photographs of this type the typical characteristics of the buds, flowers, leaves, and stems, of ‘Evipo046’. Specifically illustrated in the drawings are FIG. 1 Entire plant in flower. FIG. 2 flowers and leaves. FIG. 3 leaves, petioles, stems, and flowers.

### DETAILED DESCRIPTION OF THE VARIETY

The following is a detailed description of ‘Evipo046’, as observed in its growth throughout the flowering period in glasshouses in Denmark. Observed plants were cultivated for a period of 18 months in 2 liter containers. Certain phenotypical characteristics of the variety may vary under different environmental, cultural, agronomic, seasonal, and climatic conditions. Color references are made using The Royal Horticultural Society (London, England) Colour Chart, 2001, except where common terms of color are used.

For a comparison, several physical characteristics of the clematis variety ‘Evipo023’ described and illustrated in U.S. Plant Pat. No. 15,166 issued Sep. 21, 2004, are compared to ‘Evipo046’ in Chart 1.

CHART 1

| | ‘Evipo046’ | ‘Evipo023’ |
|---|---|---|
| flower diameter | 120 mm | 90-120 mm |
| tepal color upper, | Violet Group 85D | Violet Blue 91A |
| upon opening | | |
| anther coloration | Violet 88A | White 155A |

Flower and flower bud:
   Blooming habit.—Recurrent. The natural flowering period is generally from June to September.
   Flower bud.—Size: Normally 35 mm in length. Bud diameter is 10 mm. Bud form: Elliptic. Bud color: At ¼ opening Violet Group 88C.
   Receptacle.—Shape: Broad funnel. Size: 2 mm (h)×4 mm (w). Texture: Smooth. Color: Yellow-Green Group 144C.
   Peduncle.—Texture: Smooth, pubescent. Length: 110 mm average. Color: Yellow-Green Group 146D. Strength: Moderate.
   Flower arrangement.—Location on vine: New growth only. Borne: Singly.
   Flower bloom.—Size: On average, flowers are 120 mm in diameter and 15 mm in depth. Profile: Open flowers are flat. Fragrance: None. Lasting Quality: Flowers normally remain 7 days on the plant.
   Tepals.—Tepal Color: Upon opening, the upper surface is Violet Group 85D. The lower surface is Violet Group 85C. After opening, the upper surface is Violet Group 85B with a central bar of Violet Group 88B. The lower surface is Violet Group 85C with a central bar of White Group 155B. Quantity: Normally 7 tepals. Size: 60 mm in length by 35 mm wide. Shape: Individual tepal shape is elliptic. The tepal apex is mucronate. The tepal base is typically acute. Apex Recurvature: Slightly recurved. Tepal Cross section: Slightly. Margins: Entire. Persistence: Tepals drop off cleanly.
   Reproductive organs.—Arrangement: Moderately spreading. Pollen: Quantity: Moderate. Color: Yellow Group 8C. Anthers: Size: 5 mm in length. Color: Violet Group 88A. Quantity: On average, 100. Filaments: Color: Violet Group 88C. Basal portion is White Group 155B. Length: 5 to 12 mm. Pistils: Quantity: On average, 90. Stigmas: Inferior in location relative to the length of the filaments and the height of the anthers. Styles: Color: Yellow-Green Group 150A. Length: 4 mm.
   Seed head characteristics.—No seed observed.
Plant:
   Plant form.—Climbing.
   Plant growth.—Moderately vigorous.
   Size.—Seasons growth attains 1 meter in height. Average spread is 70 cm.
   Hardiness.—Variety hardy in USDA Zones 4-9.
   Stems.—Color: Juvenile stems are Greyed-Orange Group 164B. Mature stems are Greyed-Orange Group 164B. Internodes: On average, 10 cm between nodes. Length: Normally 50 cm from the base of the plant to the flowering portion of the stem. Diameter: Normally 2 mm. Texture: Mature stems are generally ribbed.
   Plant foliage.—Leaf characteristics: Deciduous. Arrangement: Trifoliate. Leaf Size: Compound leaves are 70-150 mm (l)×60-120 mm (w). Terminal leaflets are 50 mm (l)×40 mm (w). Abundance: On average leaves per 10 cm of stem. Leaf Color: Juvenile upper Yellow-Green Group 147D. Juvenile lower Yellow-Green Group 146B. Mature upper Yellow-Green Group 147A. Mature lower Yellow-Green Group 147B. Stipules: Absent. Petioles: Size: Normally 50-80 mm in length by 1-2 mm diameter. Texture: Smooth and pubescent. Color: Yellow-Green Group 144A. Petioloule: Size: Normally 10-15 mm in length by 1-2 mm diameter. Texture: Smooth and pubescent. Color: Greyed-Red Group 178A. Leaflet Shape: Generally lanceolate. The base is rounded. The apex is cuspidate and acute. Margin: Entire. Surface: The upper side is smooth. The lower side is smooth. Thickness: Thin. Glossiness: Not glossy.
   Disease resistance.—Subject to any disease that normally attacks the species. However the variety is more tolerant to clematis wilt than some clematis.
   Cold hardiness.—The variety is tolerant to USDA Hardiness Zone 6.
   Heat tolerance.—The variety has been found to be suitable for climate conditions found in the American Horticulture Society heat zone 7.

We claim:

1. A new and distinct variety of clematis plant named ‘Evipo046’, substantially as described and illustrated , due to its abundant violet flowers with good keepability, attractive long lasting foliage and compact growth, year round flowering under glasshouse conditions, suitability for production from softwood cuttings in pots, durable flowers and foliage which make the variety suitable for distribution in the floral industry.
